(19) United States
(12) Patent Application Publication
(10) Pub. No.: US 2018/0142073 A1
(43) Pub. Date: May 24, 2018

(54) BIODECOMPOSABLE FILM MATERIAL AND METHOD FOR MAKING THE SAME

(71) Applicant: CHIAO FU MATERIAL TECHNOLOGY CO., LTD., Taichung (TW)

(72) Inventors: Sheng-Yen Wu, Miaoli (TW); Feng-Chia Hsieh, Changhua (TW); Yuan-Fu Jhong, Taoyuan (TW); Yao-Kuei Hsiao, Taichung (TW)

(21) Appl. No.: 15/821,839

(22) Filed: Nov. 23, 2017

(30) Foreign Application Priority Data
Nov. 24, 2016 (TW) 105138718

(51) Int. Cl. C08J 5/18; C12N 1/20; C08K 3/26; C08K 3/34; C08J 7/04; B29C 47/00

(52) U.S. Cl. CPC C08J 5/18; B29K 2067/046; C08K 3/26; C08K 3/34; C08J 7/047; B29C 47/0054; B29C 47/0069; B29C 47/0011; B29C 47/0004; C08J 2367/02; C08J 2367/04; C08J 2491/00; C08J 2489/04; C08J 2403/02; C08K 2003/265; C08K 2201/003; C08J 2497/02; C08K 2201/018; C12N 1/20

(57) ABSTRACT

A biodecomposable film material includes: a biodecomposable material which is one or more selected from the group consisting of polylactic acid, poly(butylene adipate-co-terephthalate) and poly butylene succinate and which has a mass percentage of 60-70%; a food grade agricultural waste having a diameter smaller than 50 μm and a mass percentage of 10-30%; a modifier which is calcium carbonate powder or magnesium silicate salt powder and which has a diameter smaller than 8 μm and a mass percentage of 7-29%; and an organic decomposing bacterium which is Bacillus amyloliquefaciens. The organic decomposing bacterium is heat resistant bacillus capable of withstanding a temperature of 100° C. and has a mass percentage of 1-3%. The biodecomposable material, the food grade agricultural waste, the modifier, and the organic decomposing bacterium are subjected to compounding and blowing to form a film having a thickness of 40-60 μm.

### BACKGROUND OF THE INVENTION

The present invention relates to field of biodecomposable film materials and, more particularly, to a biodecomposable film material and a method for making the biodecomposable film material.

Plastic films are generally produced from refining petrochemical materials and damage the environment due to poor degradability. Many so-called “naturally degradable petrochemical materials” are merely disintegrated from large plastic particles into small plastic fragments by sunlight or chemical reaction with the chemicals in the air. These small plastic fragments produce toxins that percolate into the soil or the environmental air but not really decomposed by organisms, which are not really ecofriendly.

Thus, a need exists for a novel biodecomposable film material that can be easily decomposed and a method for making the biodecomposable film material.

### BRIEF SUMMARY OF THE INVENTION

An objective of the present invention is to provide a biodecomposable film material including: a biodecomposable material which is one or more selected from the group consisting of polylactic acid (PLA), poly(butylene adipate-co-terephthalate) (PBAT) and poly butylene succinate (PBS), wherein a mass percentage of the biodecomposable material is 60-70%; a food grade agricultural waste which is powder obtained from finely grinding starch, fibers, proteins, or lipids, wherein the food grade agricultural waste after grinding and drying has a diameter smaller than 50 μm, and wherein a mass percentage of the food grade agricultural waste is 10-30%; a modifier which is calcium carbonate (CaCO₃) powder or magnesium silicate salt powder, wherein the modifier has a diameter smaller than 8 μm, and wherein a mass percentage of the modifier is 7-29%; and an organic decomposing bacterium which is Bacillus amyloliquefaciens, wherein the organic decomposing bacterium is heat resistant bacillus capable of withstanding a temperature of 100° C., wherein a mass percentage of the organic decomposing bacterium is 1-3%, wherein the biodecomposable material, the food grade agricultural waste, the modifier, and the organic decomposing bacterium are subjected to compounding and blowing to form a film having a thickness of 40-60 μm.

Another objective of the present invention is to provide a biodecomposable film material and a method for making the biodecomposable film material. The biodecomposable film material can be decomposed by microorganisms and mainly consists of starch, fibers, proteins, or lipids, rather than petrochemical products. The raw materials of the biodecomposable film material can be absorbed by the nature without interfering with or destroying the operation of the natural environment. After decomposition by the microorganisms, the biodecomposable film material turns into natural fertilizers including carbon dioxide, water, hydrogen, oxygen, and nitrogen. The soil fertility is changed while the biodecomposable film material is decomposed, which is ecofriendly.

A further objective of the present invention is to produce a biodecomposable film material and a method for making the biodecomposable film material, which can be used as garbage bags, packaging bags, various plastic cards, diapers, agricultural materials, and drug carriers for slowing releasing drug. Furthermore, the biodecomposable film material relates to various ecofriendly plastic products, such as green nets and films for civil engineering. The biodecomposable film material can be applied to the fields of films on surfaces of packages and tableware, single-use medical articles, agricultural films, materials for slowing releasing pesticides and chemical fertilizers, and biomedical polymer materials.

Still another objective of the present invention is to provide a biodecomposable film material and a method for making the biodecomposable film material as well as addressing the problem of agricultural waste and business waste. Since the ingredients of the biodecomposable film material include powder obtained from grinding food grade agricultural waste which is, thus, finally used, fully using the resources and helping agricultural workers properly handle the agricultural waste. Furthermore, the decomposing speed of microorganisms decomposing the biodecomposable film material according to the present invention can be increased to reduce the environmental burden of handling garbage. The related industry can form a supply chain to enlarge the environmental protection level. Furthermore, during decomposition of the biodecomposable film material, the organic decomposing bacterium and the natural fertilizers (such as carbon dioxide, water, hydrogen, oxygen, nitrogen, etc.) generated by starch, plant fibers, proteins or lipids in the food grade agricultural waste can change the soil fertility to increase the utility value of farmland.

Yet another objective of the present invention is to provide a biodecomposable film material and a method for making the biodecomposable film material. Garbage is generally buried by human. After the biodecomposable film material according to the present invention comes into contact with the soil and moisture in the soil, the organic decomposing bacterium on the biodecomposable film material (by compounding or coating) is gradually released to decompose the biodecomposable film material. During decomposition of the film, the organic decomposing bacterium and the natural fertilizers (such as carbon dioxide, water hydrogen, oxygen, nitrogen) generated by the starch, plant fibers, proteins or lipids in the food grade agricultural waste can change the soil fertility to increase the utility value of farmland.

The present invention will become clearer in light of the following detailed description of illustrative embodiments of this invention described in connection with the drawings.

### DESCRIPTION OF THE DRAWINGS

FIG. 1 is a flowchart illustrating steps of a method of an example according to the present invention.

FIG. 2 is a flowchart illustrating steps of a method of another example according to the present invention.

### DETAILED DESCRIPTION OF THE INVENTION

The main ingredients of a biodecomposable film material according to the present invention is starch type biodecomposable material and starch powder, plant fiber powder, protein powder, or lipid powder, which is inferior to petrochemical material in physical properties, such as tensile strength, rigidity, degree of fusion (or polymerization), and heat resistance. Thus, a modifier (an inorganic compound) is used to modify the properties of the biodecomposable film material. The modifier used in the present invention can be calcium carbonate (CaCO₃) powder or magnesium silicate salt powder.

The raw materials used in the present invention includes polylactic acid (or polylactide, PLA), also known as cornstarch resin. The main source of PLA is starch (such as corn, beet, wheat, or sweat potato) or carbohydrate, which is fermented, dehydrated, and polymerized to produce PLA. Polylactic acid is a thermoplastic aliphatic polyester. Lactic acid and lactide, which are required for production of PLA, can be obtained from fermentation, dehydration, and purification of reusable sources. The obtained polylactic acid generally possesses excellent mechanical properties and processing performances. The discarded PLA products can rapidly degrade. Thus, polylactic acid is deemed as a green plastic material with excellent utility. The material properties, melting point, heat resistance, mechanical properties, and processing performances of polylactic acid are related to the degree of crystallinity, which is mainly affected by the ratio between L-lactic acid and D-lactic acid.

In a case that the raw material is pure L-lactic acid or pure D-lactic acid, the resultant poly-L-lactic acid (PLLA) or poly-D-lactic acid (PDLA) is a semi-crystalline polymer. PLLA has a degree of crystallinity about 37%, a glass transition temperature of about 65° C., a melting point of 180° C., a tensile modulus of about 3-4 GPA, a flexural modulus of about 4-5 GPA. The degree of crystallinity is increased significantly even though only a small amount of poly-D-lactic acid is added. For example, after mixing of poly-L-lactic acid and poly-D-lactic acid at a certain ratio, the melting point of poly-L-lactic acid can be at best increased by 50° C., and the bending temperature is increased by about 60° C. to 190° C. The resultant heat resistant PLA can be used in 110° C. environment. The mechanical properties of PLA are similar to polystyrene and polyethylene terephthalate (PETE), but PLA has a lower continuous use temperature. The continuous use temperature is increased when the degree of crystallinity is increased, but the biodegrading speed is reduced.

When placed in a crucible and heated, PLA vaporizes into a flammable gas. In comparison with other biodegradable materials, PLA has partial hydrophilicity, The best solvent for PLA and copolymers of PLA is chloroform. Furthermore, PLA can dissolve in chlorinated solvent, hot benzene, tetrahydrofuran, and 1,4-dioxane but cannot dissolve in water, ethanol, and most of aliphatic hydrocarbon solvents.

Poly(butylene adipate-co-terephthalate) (PBAT) is a thermoplastic biodegradable plastic material and is a copolymer of polybutylene adipate (PBA) and polybutylene terephthalate (PBT) and, thus, includes the characteristics of both PBA and PBT. Thus, PBAT has better heat resistance and better impact resistance. Furthermore, PBAT has excellent biodegradability and is one of the best degrading materials in the market in the reaches of biodegrading materials. PBAT is a semi-crystalline copolymer having a crystallization temperature of about 110° C., a melting point of 130° C., and a density between 1.18-1.3 g/ml. The degree of crystallinity of PBAT is about 30%, and the Shore hardness of PBAT is above 85. PBAT is a copolymer of aliphatic and aromatic polyesters and, thus has the excellent biodegrading performance of aliphatic polyester and the excellent kinetic performance of aromatic polyester as well as sufficient toughness.

Poly butylene succinate (PBS) is a thermoplastic biodegradable plastic material and is a copolymer of succinic acid and 1,4-butanediol. PBS entered the research field in 1990s, is cheap, and has excellent resistance to heat. The chemical formula of PBS is HO—(CO—(CH₂)₂—CO—O—(CH₂)₄—O)ₙH. PBS is in the form of white particles. PBS is obtained from condensation, polymerization, and synthesis of butanedioic acid and butanediol. The resin is milky white, odorless, and tasteless. PBS is apt to be decomposed and metabolized by enzymes in various microorganisms, animals, and plants and is finally decomposed into carbon dioxide and water, which is a typical completely biodegradable polymer material with excellent biocompatibility and bio-absorbability.

Calcium carbonate is an organic compound and is also known as gray stone, limestone, stone powder, marble, and calcite. Calcium carbonate is a compound with a chemical formula of CaCO₃ and is alkaline. Generally, calcium carbonate does not dissolve in water but dissolves in acid. Calcium carbonate is an ordinary substance existing in aragonite, calcite, chalk, limestone, marble, and calcareous tufa. Calcium carbonate is widely used in filling resins and plastic materials. Adding calcium carbonate improves some performances of plastic products to increase their applications. In plastic material processing, calcium carbonate reduces the contraction coefficient of resin, improves the flow transition, and controls the viscosity.

Calcium carbonate also provides the following functions:

1. Improving Stability of Size of Plastic Products: Addition of calcium carbonate provides a skeleton effect, which greatly helps stabilization of the size of plastic products.

2. Increasing Hardness and Rigidity of Plastic Products: Calcium carbonate generally cannot provide an enhancing function in plastic materials and resins. Nevertheless, the particles of calcium carbonate can be immersed in resins. Thus, addition of calcium carbonate not only increases the rigidity of plastic materials and resins but also increases the elastic modulus and hardness. As the amount of added calcium carbonate increases, the tensile strength and the elongation limit decrease. The hardness varies as the type of calcium carbonate and the amount of added calcium carbonate change.

3. Improving Plastic Material Processing Performance: Addition of calcium carbonate changes the flow transition performances of plastic materials. A larger amount of calcium powder is generally added to improve mixing with other ingredients and to enhance formation of plastic materials. Addition of calcium carbonate, particularly surface-treated calcium carbonate, not only increases the hardness of the products but improves surface glossiness and surface evenness of the products, reducing the contraction coefficient, linear expansion coefficient and creep performance of the plastic product, thereby creating processing conditions.

4. Increasing Heat Resistance of Plastic Products: Addition of calcium carbonate into ordinary plastic products increases the heat resistance. For example, addition of about 40% of calcium carbonate into polypropylene increases the heat resistance by about 200° C. When the filling ratio is not larger than 20%, the heat resistance is increased by 8-130° C.

Magnesium silicate salt is talcum powder whose main component is talc, water-containing magnesium silicate with a chemical formula of Mg₃[Si₄O₁₀](OH)₂. The physical and chemical properties include lubricity, resistance to adhesion, flow aiding performance, resistance to fire, resistance to acid, insulativity, high melting point, inactive chemical properties, excellent covering properties, soft, glossiness, and excellent absorbability. Since talc has a layer-like crystalline structure, talc is liable to break into scales, has special lubricity, and is applied in chemical industries (such as rubber, plastic, paint) as a reinforcing, modifying, filling agent. It can increase the shape stability, tensile strength, shear strength, flexing strength, compressive resistance, can reduce deformation, elongation, heat expansion coefficient, and has high whiteness, uniform particle sizes, and high dispersibility.

The organic decomposing bacterium is Bacillus amyloliquefaciens which has a better bacterium inhibiting effect and which includes bacterial strains with decomposing abilities of at least four enzymes including protein decomposing enzyme, lipid decomposing enzyme, starch decomposing enzyme, and fiber decomposing enzyme. Thus, the organic decomposing bacterium can decompose PLA, PBAT, PBS, and the food grade agricultural waste, no matter the food grade agricultural waste is starch, fiber, protein, or lipid. The organic decomposing bacterium is heat resistant bacillus capable of withstanding a temperature of 100° C.

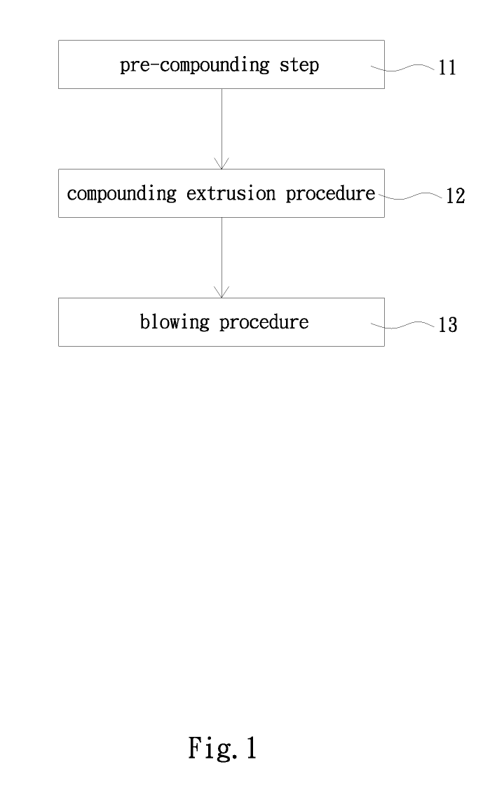

With reference to FIG. 1, a biodecomposable film material of an example according to the present invention includes:

a biodecomposable material which is one or more selected from the group consisting of polylactic acid (PLA), poly(butylene adipate-co-terephthalate) (PBAT) and poly butylene succinate (PBS), wherein a mass percentage of the biodecomposable material is 60-70%;

a food grade agricultural waste which is powder obtained from finely grinding starch, fibers, proteins, or lipids, wherein the food grade agricultural waste after grinding and drying has a diameter smaller than 50 μm, and wherein a mass percentage of the food grade agricultural waste is 10-30%;

a modifier which is calcium carbonate (CaCO₃) powder or magnesium silicate salt powder, wherein the modifier has a diameter smaller than 8 and wherein a mass percentage of the modifier is 7-29%; and

an organic decomposing bacterium which is Bacillus amyloliquefaciens, wherein the organic decomposing bacterium is heat resistant bacillus capable of withstanding a temperature of 100° C., wherein a mass percentage of the organic decomposing bacterium is 1-3%, wherein the biodecomposable material, the food grade agricultural waste, the modifier, and the organic decomposing bacterium are subjected to compounding and blowing to form a film having a thickness of 40-60 μm.

The food grade agricultural waste can be one or more selected from the group consisting of distiller's grain powder, brewery spent grain powder, rice straw powder, bean shell powder, and coffee grounds powder.

The food grade agricultural waste can be an agricultural waste that passes pesticide residue test and heavy metal residue test. Namely, the agricultural waste is free of pesticide residue and heavy metal residue.

The film having the thickness of 40-60 μm can be changed to match with the period of time of biodecomposition.

A method for making a biodecomposable film material of an example according to the present invention includes:

a pre-compounding step 11 including increasing a temperature of a compounding extruder to 150-170° C.;

a compounding extrusion procedure 12 including compounding a mixture of a biodecomposable material of 60-70 m %, a food grade agricultural waste of 10-30 m % and having a diameter smaller than 50 μm and having a diameter smaller than 8 μm, a modifier of 7-29 m %, and Bacillus amyloliquefaciens of 1-3 m % at 150-170° C. for not more than 10 seconds and then extruding the mixture into particles; and

a blowing procedure 13 including blowing the particles into a thin film at 150-170° C. for not more than 10 seconds, wherein the film contains viable bacteria with a concentration lower than 9.5×10¹ CFU/cm² and has a standard thickness of 40-60 μm after taking shape.

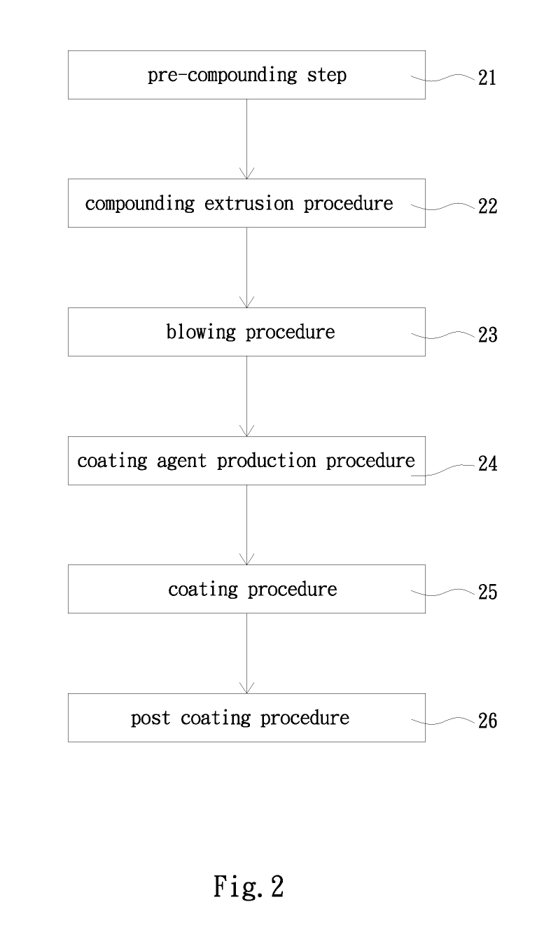

With reference to FIG. 2, a biodecomposable film material of another example according to the present invention includes:

a biodecomposable material which is one or more selected from the group consisting of polylactic acid (PLA), poly(butylene adipate-co-terephthalate) (PBAT) and poly butylene succinate (PBS), wherein a mass percentage of the biodecomposable material is 20-50%;

a food grade agricultural waste which is powder obtained from finely grinding starch, fibers, proteins, or lipids, wherein the food grade agricultural waste after grinding and drying has a diameter smaller than 50 μm, and wherein a mass percentage of the food grade agricultural waste is 10-30%;

a modifier which is calcium carbonate (CaCO₃) powder or magnesium silicate salt powder, wherein the modifier has a diameter smaller than 8 μm, and wherein a mass percentage of the modifier is 10-30%; wherein the modifier is subjected to compounding and blowing to form a film having a thickness of 40-60 μm; and

a coating agent obtained from uniformly stirring adhesive starch, water, and an organic decomposing bacterium, wherein the organic decomposing bacterium is Bacillus amyloliquefaciens which is heat resistant bacillus capable of withstanding a temperature of 100° C., wherein the coating agent includes 9.8-19.8 m % of starch, 80-90 m % of water, and 0.2 m % of organic decomposing bacterium, wherein the organic decomposing bacterium is diluted by a 1/500 dilution, the coating agent is uniformly coated by a coating method on an upper face and a lower face of the film, wherein the film is dried naturally, and wherein the film contains viable bacteria with a concentration lower than 7×10² CFU/cm².

The unit CFU/cm² means the average number of bacterial strains of bacillus per square centimeter.

The coating method can be selected from the group consisting of spray coating, brush coating, printing coating, and dip coating.

When carrying out the printing coating, the coating agent includes 80-90 m % of starch, 10-20 m % of water, and 1-3 m % of organic decomposing bacterium.

The food grade agricultural waste can be one or more selected from the group consisting of distiller's grain powder, brewery spent grain powder, rice straw powder, bean shell powder, and coffee grounds powder.

The food grade agricultural waste is an agricultural waste that passes pesticide residue test and heavy metal residue test. Namely, the agricultural waste is free of pesticide residue and heavy metal residue.

The film having the thickness of 40-60 μm can be changed to match with the period of time of biodecomposition.

A method for making a biodecomposable film material of another example according to the present invention includes:

a pre-compounding step 21 including increasing a temperature of a compounding extruder to 150-170° C.;

a compounding extrusion procedure 22 including compounding a mixture of a biodecomposable material of 20-50 m %, a food grade agricultural waste of 10-30 m % and having a diameter smaller than 50 μm, and a modifier of 10-30 m % and having a diameter smaller than 8 μm at 150-170° C. for not more than 10 seconds and then extruding the mixture into particles;

a blowing procedure 23 including blowing the particles into a film at 150-170° C. for not more than 10 seconds, wherein the film has a standard thickness of 40-60 μm after taking shape;

a coating agent production procedure 24 including preparing a coating agent that includes 0.2 m % of organic decomposing bacterium, 9.8-19.8 m % of starch, and 80-90 m % of water, wherein the organic decomposing bacterium is Bacillus amyloliquefaciens, wherein the organic decomposing bacterium is diluted by a 1/500 dilution;

a coating procedure 25 including uniformly coating the coating agent on an upper face and a lower face of the film by spray coating, brush coating, printing coating, and dip coating;

a post coating procedure 26 including naturally drying the film and detecting the film after taking shape, wherein the film contains viable bacteria with a concentration lower than 7×10² CFU/cm².

When carrying out the printing coating, the coating agent includes 80-90 m % of starch, 10-20 m % of water, and 1-3 m % of organic decomposing bacterium.

Although specific embodiments have been illustrated and described, numerous modifications and variations are still possible without departing from the scope of the invention. The scope of the invention is limited by the accompanying claims.

1. A biodecomposable film material comprising:
a biodecomposable material which is one or more selected from the group consisting of polylactic acid (PLA), poly(butylene adipate-co-terephthalate) (PBAT) and poly butylene succinate (PBS), wherein a mass percentage of the biodecomposable material is 60-70%;
a food grade agricultural waste which is powder obtained from finely grinding starch, fibers, proteins, or lipids, wherein the food grade agricultural waste after grinding and drying has a diameter smaller than 50 μm, and wherein a mass percentage of the food grade agricultural waste is 10-30%;
a modifier which is calcium carbonate (CaCO₃) powder or magnesium silicate salt powder, wherein the modifier has a diameter smaller than 8 μm, and wherein a mass percentage of the modifier is 7-29%; and
an organic decomposing bacterium which is Bacillus amyloliquefaciens, wherein the organic decomposing bacterium is heat resistant bacillus capable of withstanding a temperature of 100° C., wherein a mass percentage of the organic decomposing bacterium is 1-3%, wherein the biodecomposable material, the food grade agricultural waste, the modifier, and the organic decomposing bacterium are subjected to compounding and blowing to form a film having a thickness of 40-60 μm.

2. The biodecomposable film material as claimed in claim 1, wherein the food grade agricultural waste is one or more selected from the group consisting of distiller's grain powder, brewery spent grain powder, rice straw powder, bean shell powder, and coffee grounds powder.

3. The biodecomposable film material as claimed in claim 1, wherein the food grade agricultural waste is an agricultural waste free of pesticide residue and heavy metal residue.

4. The biodecomposable film material as claimed in claim 1, wherein the film having the thickness of 40-60 μm is changeable to match with a period of time of biodecomposition.

5. A method for making a biodecomposable film material, comprising:
a pre-compounding step including increasing a temperature of a compounding extruder to 150-170° C.;
a compounding extrusion procedure including compounding a mixture of a biodecomposable material of 60-70 m %, a food grade agricultural waste of 10-30 m % and having a diameter smaller than 50 μm and having a diameter smaller than 8 μm, a modifier of 7-29 m %, and Bacillus amyloliquefaciens of 1-3 m % at 150-170° C. for not more than 10 seconds and then extruding the mixture into particles; and
a blowing procedure including blowing the particles into a thin film at 150-170° C. for not more than 10 seconds, wherein the film contains viable bacteria with a concentration lower than 9.5×10¹ CFU/cm² and has a standard thickness of 40-60 μm after taking shape.

6. A biodecomposable film material comprising:
a biodecomposable material which is one or more selected from the group consisting of polylactic acid (PLA), poly(butylene adipate-co-terephthalate) (PBAT) and poly butylene succinate (PBS), wherein a mass percentage of the biodecomposable material is 20-50%;
a food grade agricultural waste which is powder obtained from finely grinding starch, fibers, proteins, or lipids, wherein the food grade agricultural waste after grinding and drying has a diameter smaller than 50 μm, and wherein a mass percentage of the food grade agricultural waste is 10-30%;
a modifier which is calcium carbonate (CaCO₃) powder or magnesium silicate salt powder, wherein the modifier has a diameter smaller than 8 μm, and wherein a mass percentage of the modifier is 10-30%; wherein the modifier is subjected to compounding and blowing to form a film having a thickness of 40-60 μm; and
a coating agent obtained from uniformly stirring adhesive starch, water, and an organic decomposing bacterium, wherein the organic decomposing bacterium is Bacillus amyloliquefaciens which is heat resistant bacillus capable of withstanding a temperature of 100° C., wherein the coating agent includes 9.8-19.8 m % of starch, 80-90 m % of water, and 0.2 m % of organic decomposing bacterium,
wherein the organic decomposing bacterium is diluted by a 1/500 dilution, the coating agent is uniformly coated by a coating method on an upper face and a lower face of the film, wherein the film is dried naturally, and wherein the film contains viable bacteria with a concentration lower than 7×10² CFU/cm².

7. The biodecomposable film material as claimed in claim 6, wherein the coating method is selected from the group consisting of spray coating, brush coating, and dip coating.

8. The biodecomposable film material as claimed in claim 6, wherein the food grade agricultural waste is one or more selected from the group consisting of distiller's grain powder, brewery spent grain powder, rice straw powder, bean shell powder, and coffee grounds powder.

9. The biodecomposable film material as claimed in claim 6, wherein the food grade agricultural waste is an agricultural waste free of pesticide residue and heavy metal residue.

10. The biodecomposable film material as claimed in claim 6, wherein the film having the thickness of 40-60 μm is changeable to match with a period of time of biodecomposition.

11. A method for making a biodecomposable film material, comprising:
a pre-compounding step including increasing a temperature of a compounding extruder to 150-170° C.;
a compounding extrusion procedure including compounding a mixture of a biodecomposable material of 20-50 m %, a food grade agricultural waste of 10-30 m % and having a diameter smaller than 50 μm, and a modifier of 10-30 m % and having a diameter smaller than 8 μm at 150-170° C. for not more than 10 seconds and then extruding the mixture into particles;
a blowing procedure including blowing the particles into a film at 150-170° C. for not more than 10 seconds, wherein the film has a standard thickness of 40-60 μm after taking shape;
a coating agent production procedure including preparing a coating agent that includes 0.2 m % of organic decomposing bacterium, 9.8-19.8 m % of starch, and 80-90 m % of water, wherein the organic decomposing bacterium is Bacillus amyloliquefaciens, wherein the organic decomposing bacterium is diluted by a 1/500 dilution;
a coating procedure including uniformly coating the coating agent on an upper face and a lower face of the film by spray coating, brush coating, and dip coating;
a post coating procedure including naturally drying the film and detecting the film after taking shape, wherein the film contains viable bacteria with a concentration lower than 7×10² CFU/cm².

12. A biodecomposable film material comprising:
a biodecomposable material which is one or more selected from the group consisting of polylactic acid (PLA), poly(butylene adipate-co-terephthalate) (PBAT) and poly butylene succinate (PBS), wherein a mass percentage of the biodecomposable material is 20-50%;
a food grade agricultural waste which is powder obtained from finely grinding starch, fibers, proteins, or lipids, wherein the food grade agricultural waste after grinding and drying has a diameter smaller than 50 μm, and wherein a mass percentage of the food grade agricultural waste is 10-30%;
a modifier which is calcium carbonate (CaCO₃) powder or magnesium silicate salt powder, wherein the modifier has a diameter smaller than 8 and wherein a mass percentage of the modifier is 10-30%, wherein the modifier is subjected to compounding and blowing to form a film having a thickness of 40-60 μm; and
a coating agent obtained from uniformly stirring adhesive starch, water, and an organic decomposing bacterium, wherein the organic decomposing bacterium is Bacillus amyloliquefaciens which is heat resistant bacillus capable of withstanding a temperature of 100° C., wherein the coating agent includes 80-90 m % of starch, 10-20 m % of water, and 1-3 m % of organic decomposing bacterium, wherein the coating agent is uniformly coated an upper face and a lower face of the film by printing coating, and the film is dried naturally, wherein the film contains viable bacteria with a concentration lower than 7×10² CFU/cm².

13. A method for making a biodecomposable film material, comprising:
a pre-compounding step including increasing a temperature of a compounding extruder to 150-170° C.;
a compounding extrusion procedure including compounding a mixture of a biodecomposable material of 20-50 m %, a food grade agricultural waste of 10-30 m % and having a diameter smaller than 50 μm, and a modifier of 10-30 m % and having a diameter smaller than 8 μm at 150-170° C. for not more than 10 seconds and then extruding the mixture into particles;
a blowing procedure including blowing the particles into a film at 150-170° C. for not more than 10 seconds, wherein the film has a standard thickness of 40-60 μm after taking shape.
a coating agent production procedure including preparing a coating agent that includes 1-3 m % of organic decomposing bacterium, 80-90 m % of starch, and 10-20 m % of water, wherein the organic decomposing bacterium is Bacillus amyloliquefaciens;
a coating procedure including uniformly coating the coating agent on an upper face and a lower face of the film by printing coating; and
a post coating procedure including naturally drying the film and detecting the film after taking shape, wherein the film contains viable bacteria with a concentration lower than 7×10² CFU/cm².
